(19) United States
(12) Patent Application Publication
(10) Pub. No.: US 2008/0118448 A1
(43) Pub. Date: May 22, 2008

(54) Colored Metal Pigments, Method for the Production Thereof, Use of the Colored Metal Pigments in Cosmetics, and Cosmetic Containing These

(76) Inventor: Peter Krüger, Schnaittach (DE)

Correspondence Address: OSTROLENK FABER GERB & SOFFEN, 1180 AVENUE OF THE AMERICAS, NEW YORK, NY, 100368403, US

(21) Appl. No.: 10/584,695

(22) PCT Filed: Dec. 15, 2004
(86) PCT No.: PCT/DE04/02742

§ 371 Date: Aug. 16, 2006

(30) Foreign Application Priority Data
Dec. 23, 2003 (DE) 103 61 436.2

(51) Int. Cl. C04B 14/30; A61K 8/00; B05D 5/06; A61Q 17/04; A61Q 1/06; A61Q 1/00; A61Q 19/00; A61Q 5/06; C04B 16/00

(52) U.S. Cl. 424/59; 106/415; 427/216; 424/63; 424/61; 424/70.7

(57) ABSTRACT

The invention relates to a colored, platelet-shaped metal pigment comprising a base pigment comprising metal, a layer of silicon dioxide, and, subsequently, a layer or two or more layers comprising one or more coloring materials. The invention further relates to a method for the production of a colored metal pigment of this kind, and also to the use thereof in cosmetics.

The present invention relates to colored metal pigments, methods for the production thereof, the use of the colored metal pigments in cosmetics, and a cosmetic produced using the colored metal pigments.

Cosmetics serve to protect skin, hair, finger- and toenails, eyes, and lips from mechanical damage, drying out, and infections. In many cases, however, besides the care and protection aspect, the intention is also to obtain a particular visual effect, by addition of coloring and effect-imparting agents, for example.

These coloring and effect-imparting agents may be dyes, laked organic dyes, organic or inorganic pigments or effect pigments, the primary desire particularly in the case of effect pigments being to obtain a different perceived color or perceived brightness depending on the angle from which the applied product is viewed. Pearlescent pigments in particular are employed conventionally for this purpose in the cosmetics sector.

By effect pigments are meant pigments which exhibit directed reflection at predominantly two-dimensional, oriented metallic or highly refractive particles (DIN 55944). They are always platelet-shaped and, in comparison with color pigments, have very large particle diameters. Their optical properties are determined by reflection and interference. Depending on transparency, absorption, thickness, single-layer and multilayer construction, the effect pigments display metallic gloss, pearlescence, interference, or interference reflection. Principal application sectors are automotive finishes, coloring of plastics, paints, leather coatings, printing inks, and coloring of ceramics. Pearlescent pigments are much employed in cosmetics. For a comprehensive depiction of the technical background cf. W. Ostertag, Nachr. Chem. Tech. Lab. 1994, 9, 849.

The most frequently used effect pigments are aluminum flakes and coated mica platelets, with aluminum pigments displaying typical metal gloss and coated mica platelets typical pearlescence.

In recent years there has been a sharp increase in demand for colored effect pigments. Consequently, for example, oxide-coated copper and brass platelets, substrates such as muscovite, phlogopite or glass coated with transition metal oxides, guanine monocrystals (pearl essence), BiOCl monocrystals, platelet-shaped hematite monocrystals; platelet-shaped phthalocyanines or comminuted thin multilayer films with Fabry-Perot structure have been used as effect pigments.

In order to obtain color effects, pigments, including for example aluminum pigments, are mixed with transparent color pigments. With this technique, however, the coloristic possibilities are limited insofar as no interference effects can be obtained in this way; in other words, the pigments do not exhibit pearlescence. Colored interference pigments with pearlescence, mostly based on coated mica platelets, in turn have a poorer opacity, owing to their transparency, than aluminum pigments. As a consequence of this there has been no lack of attempts to produce, by coloring aluminum pigments, pigments having the good opacity of the aluminum flakes and the coloristic possibilities of the interference pigments.

U.S. Pat No. 4,328,042 and EP-A-0033457 describe the production of gold-colored pigments by deposition of iron oxide on aluminum, with iron pentacarbonyl being oxidized with oxygen in a fluidized bed of the aluminum flakes generated by fluidization with inert gas. The disadvantage of this method is the very great technological cost and complexity.

U.S. Pat. No. 5,037,475 describes the production of aluminum pigments colored by fixing color pigments on the metal surface. The color pigments are fixed by way of carboxyl-containing polymers. For enhancing adhesion it is possible to apply a protective layer by polymerization. The color intensity of the pigments thus produced, however, is low.

WO 91/04293 (PCT/US90/05236) describes the coloring of aluminum pigments by fixing of polymer-coated color pigments from aqueous solvents by way of electrostatic forces on the metal surfaces. The coating outcome depends in this case, in a complex way, on the type of aluminum pigment, the nature of the polymeric coating of the color pigments, the solvent composition, and the pH.

EP-A-0 328 906 discloses titanium dioxide-coated metal pigments, including aluminum pigments, which are produced by hydrolyzing an organic titanate ester compound, tetraisopropoxytitanium for example, in the presence of the metal flakes, in suspension in an organic medium, at a pH of 4 to 8.

Different hues can be achieved with this method by varying the thickness of the titanium dioxide coat. For the production of the coated pigments, compliance with specific conditions is critical. The pH must lie within the range from 4 to 8, and the rate of dropwise addition when adding the titanate ester must lie within the range from 1.0×10⁻⁷ to 1.0×10⁻⁴ mol per minute per m² of metal surface. As a consequence of this the method is virtually impossible to employ on the industrial scale. Moreover, after the coated pigments have been dried they must be calcined in order to obtain color effects, since only by removing the water from the metal oxide coat is a suitable coat structure developed. Owing to the low melting point of aluminum, however, the calcining of coated aluminum pigments is possible only with great difficulty.

U.S. Pat. No. 4,978,394 describes the production of titanium dioxide-coated aluminum pigments by chemical gas-phase coating (chemical vapor deposition, CVD), with titanium tetrachloride being reacted at a low concentration with water vapor in a fluidized bed in the presence of hot aluminum particles. With this method, too, the costly and complicated technology has deleterious consequences.

U.S. Pat. No. 4,158,074 discloses the production of colored aluminum pigments by coating with a film of hydrated aluminum oxide and hydrated metal oxide. The film is produced by treating fine aluminum flakes in an alkaline solution of a salt of iron, of nickel, of cobalt, of zinc or of copper at an elevated temperature and at a pH of 8 to 12; in other words, by an electro-chemical reaction of the metal salts. In this way it is possible to produce gold-colored pigments, and also, by adding chelating agents, black-brown and gray-white pigments. Accordingly there is only a narrowly confined color range available.

DE-C 25 57 796 discloses colored metal pigments coated with a metal oxide layer comprising carbon black as color pigment. The pigments disclosed are either jet black pigments or colored pigments with particular color effects.

DE-A 36 17 430 describes pearlescent pigments composed of platelet-shaped colored pigments with a layer containing iron oxide. Glass platelets, metal flakes, and graphite are disclosed as platelet-shaped substrates. The layer containing iron(II) oxide may be present as a simple iron(II) oxide layer or as a layer of a mixed oxide such as iron aluminate, chromite, iron orthosilicate, and also ilmenite.

DE-A 42 23 383 relates to luster pigments with a coating containing metal sulfide and based on coated platelet-shaped silicatic or metallic substrates. Metallic substrates disclosed are all flake-formed metals known for metallic effect pigments, especially copper and its alloys, such as brass or bronzes, and particularly aluminum and its alloys such as aluminum bronzes. To produce chromatic pigments exhibiting interference colors the substrate particles may already be coated with a layer of high-refractive-index metal oxide, on which the layer containing metal sulfide is applied to the substrate preferably by the gas phase.

DE-A 42 23 384 discloses luster pigments based on metal oxide-coated aluminum flakes, having a total carbon content of <0.1% by weight. Preferred oxides mentioned for the metal oxide layer are titanium oxide, zirconium oxide, tin oxide, and chromium oxide, and iron oxide as well.

JP-1-110568 discloses a colored aluminum powder pigment obtained by hydrolyzing a metallic alkoxide (e.g., tetraethyl titanate), coating the surface of an aluminum powder with the resulting product, and further heating the resultant powder to a temperature of >200° C. and below the ignition point of the aluminum powder.

U.S. Pat. No. 5,261,955 describes a sol-gel process for producing colored metal pigments, the metal flakes being dispersed in a sol of an inorganic salt, such as an aqueous alkaline zirconium oxide sol, for example, dispersing the sol-coated flakes, following filtration, in a solution of an inorganic compound, cobalt nitrate for example, in an organic solvent, and heating to form a sol-gel layer on the flakes. As a result of the multiplicity of individual steps this method too requires a high level of complexity in terms of apparatus.

JP-A-61-1 30375 discloses a gold-colored aluminum pigment produced by treating aluminum powder with dichromate, sodium fluoride, and surface-active agents in acidic solution, drying, and subsequently treating with a fatty acid derivative. Hues other than gold cannot be obtained by this technique.

DE-A 41 40 295 describes pigments comprising platelet-shaped carrier materials, preferably mica, which are coated with an inorganic matrix comprising metal oxides and/or dye particles in the submicrometer range. The substrates are coated from acidic aqueous suspensions by hydrolysis of metal salts, preferably titanium tetrachloride, in the presence of particles of metal oxide and/or of dye. The coloring of aluminum flakes by this method, however, is not possible, because under these conditions the aluminum particles are rapidly decomposed.

U.S. Pat. No. 3,067,052 discloses colored aluminum pigments produced by oxidizing aluminum powder with KMnO₄ solution, with addition, if desired, of a reducing agent. The hue of these pigments is golden, possibly also with a greenish or reddish shade depending on the reducing agent used.

With the methods for the production of colored, mica-based pigments it is not possible, for the reasons stated, to produce colored aluminum pigments, although it is these, owing to the relatively high opacity and the metal luster, that are of great interest. The known methods of coloring aluminum pigments, however, produce only a few hues, predominantly in the gold region, and are very largely highly complex in terms of apparatus. As a consequence there continues to be a demand for aluminum pigments colored in different hues and for a method, employing simple apparatus, for the production of these colored aluminum pigments.

Owing to existing laws, the prior art pigments generally cannot be employed in cosmetic products.

The object of the present invention is to provide colored metal pigments of this kind having preferably a good opacity and being preferably suitable for use in cosmetics.

The object on which the invention is based is achieved in accordance with the invention through the provision of a colored, platelet-shaped metal pigment comprising a base pigment comprising metal, a layer of silicon dioxide, and, subsequently, a layer or two or more layers comprising one or more coloring materials.

Preferred developments are specified in dependent claims 2 to 10.

The pigments of the invention can also be combined effectively in conjunction with other platelet-shaped effect pigments, such as pearlescent pigments, metal pigments, for example, or with organic and inorganic absorption color pigments.

The object is further achieved by provision of a method of producing a colored metal pigment, comprising the following steps:
a. coating a base pigment with SiO₂,
b. dispersing the metal pigment coated in step (a) in a liquid phase, preferably water,
c. adding one or more solutions of one or more metal salts for the purpose of generating coloring materials,
d. separating off the colored metal pigment obtained in step (c), and
e. optionally drying the colored metal pigment obtained in step (d).

Preferred developments are specified in dependent claims 12 to 15.

The liquid phase used is preferably water. It will be appreciated that organic solvents or aqueous organic solvents can also be used.

In step (c) the pH is preferably set in the range from 1.7 to 9.5. In the course of coloration the pH is preferably kept constant, which can be effected by metered addition of a suitable acid or base.

The object on which the invention is based is further achieved through the use of the colored metal pigments of the invention in cosmetics, and also through a cosmetic which comprises the colored metal pigments of the invention.

The invention accordingly provides cosmetic preparations which comprise the colored metal pigments of the invention. Suitable compositions for the cosmetic preparations into which the colored metal pigments of the invention can be introduced are known to the skilled worker.

The formulation of cosmetics using the metal pigments of the invention is accomplished by measures and methods familiar to the skilled worker.

The pigments of the invention are particularly suitable for use in lipsticks, eyecare compositions, such as eyeshadows, mascara, eyeliners, creams, powders, perfumes, nail varnishes, body sprays, makeup formulations, haircare compositions, soap or sun protection compositions.

The pigments of the invention are used preferably in amounts of 1% to 50% by weight, preferably 2%-30% by weight, and more preferably of 2.5%-15% by weight, based in each case on the total weight of the cosmetic formulation, in cosmetics.

The pigments of the invention comprise a platelet-shaped metallic base pigment with a coating of silicon dioxide and also one or more subsequent coatings which comprise one or more coloring materials.

The metallic base pigment is composed for example of aluminum, copper, zinc, iron, gold bronze, silver or alloys thereof.

The base pigment is optionally coated, with for example a layer of aluminum oxide (product range ALOXAL from Eckart GmbH & Co. KG).

The coloring material may be an inorganic metal oxide such as FeOOH, Fe₂O₃ or Cr₂O₃, and may also be Prussian Blue or carmine red.

The coloring material may be present alone, in mixtures or successive layers.

On the initial pigment there is preferably a passivating layer of silicon dioxide and, subsequently, one or more layers of coloring material.

The pigments of the invention are produced by coating platelet-shaped metal pigments with silicon dioxide, preferably by hydrolyzing organic or inorganic silicon compounds in a sol-gel process. Coating can also take place, however, with waterglass.

Methods of this kind are described for example in EP 1 084 198 and U.S. Pat. No. 2,885,366, hereby incorporated by reference.

Metal pigments coated with silicon dioxide are available commercially. Specifically for the cosmetics sector they are offered under the name Visionaire Metallic Pigments (Eckart GmbH & Co. KG).

The metal pigments coated with silicon dioxide are coated with colored metal oxides, Prussian Blue, carmine red, and mixtures of these substances by methods that are known per se.

A deep blue pigment can be obtained by precipitated application of iron(III) hexacyanoferrate(II). For this purpose an aqueous iron(III) chloride solution and an aqueous potassium hexacyanoferrate(II) solution are metered at a pH of 1.8 to 4, simultaneously, into a dispersion, heated at 60 to 90° C., of the silicon dioxide-coated metal pigment. The pigment can be worked up conventionally and dried at 80 to 130° C. It is likewise possible, by common techniques, first to deposit colorless iron(II) hexacyanoferrate(II) and subsequently to carry out oxidation to Prussian Blue.

In the case of coating with yellow iron oxide in the goethite modification it is possible to start from an iron(III) salt solution, which is metered at a pH of 2.5-4.5 into a suspension, heated at 60-90° of the metal pigment, the pH being kept constant by simultaneous addition of aqueous sodium hydroxide solution.

A green pigment can be obtained by coating the silicon dioxide-coated metal pigment with chromium oxide. Coating takes place by metered addition of a chromium chloride solution to a suspension of the metal pigments, heated at 60 to 90° C., at a pH of 5.5-9, it being possible after conventional workup to subject the pigment to heat treatment at 500-660° C.

A red pigment can be accomplished by metering a carmine solution, adjusted to a pH of 11, into a silicon dioxide-coated metal pigment coated with aluminum hydroxide, at a pH of 5.5.

Set out hereinbelow are examples of colored, stabilized metal pigments and their use in cosmetics. These examples are intended to illustrate further the invention, but not to restrict it.

### Example 1

### Coating of Aluminum Flakes With Prussian Blue

200 g of SiO₂-coated aluminum pigment (Visionaire Bright Silver Sea from ECKART GmbH & Co. KG) are dispersed with stirring in 1000 g of fully demineralized water and the dispersion is heated to 75° C. and adjusted to a pH of 4 using 10% strength hydrochloric acid. Then a solution of 42.75 g of iron(III) chloride in 240 g of water and a solution of 50.4 g of potassium hexacyanoferrate(II) in 240 g of water are added, simultaneously but separately from one another, and the pH is kept constant by simultaneous addition of 5% strength hydrochloric acid or NaOH. The metering rates are 2 ml/min. Addition of the two solutions is followed by stirring for 30 min. The solid is separated off on a suction filter, washed free of salt with fully demineralized water, and dried in vacuo at 60° C.

This gives a blue, lustrous powder.

### Example 2

### Coating of Gold Bronze Pigments With Prussian Blue

200 g of SiO₂-coated gold bronze pigment (Visionaire Bright Natural Gold from ECKART GmbH & Co. KG) are dispersed with stirring in 1000 g of fully demineralized water and the dispersion is heated to 75° C. and adjusted to a pH of 4 using 10% strength hydrochloric acid. Then a solution of 42.75 g of iron(III) chloride in 240 g of water and a solution of 50.4 g of potassium hexacyanoferrate(II) in 240 g of water are added, simultaneously but separately from one another, and the pH is kept constant by simultaneous addition of 5% strength hydrochloric acid or NaOH. The metering rates are 2 ml/min. Addition of the two solutions is followed by stirring for 30 min. The solid is separated off on a suction filter, washed free of salt with fully demineralized water, and dried in vacuo at 60° C.

This gives a green, lustrous powder.

### Example 3

### Coating of Aluminum Pigments With Carmine

200 g of SiO₂-coated aluminum pigment (Visionaire Bright Silver Sea from ECKART GmbH & Co. KG) are dispersed with stirring in 1000 g of fully demineralized water and the dispersion is heated to 75° C. and adjusted to a pH of 6 using 10% strength hydrochloric acid. Then a solution of 9.32 g of aluminum(III) chloride in 120 g of water is added. The metering rate is 8 ml/min. The pH is kept constant by addition of 5% strength aqueous sodium hydroxide solution. Subsequently 13.6 g of carmine lake in 800 ml of fully demineralized water are introduced. The metering rate is 8 ml/min. Addition of the two solutions is followed by stirring for 30 min. The solid is separated off on a suction filter, washed free of salt with fully demineralized water, and dried in vacuo at 80° C.

This gives a red, lustrous powder.

### Example 4

### Coating of Gold Bronze Pigments With Carmine

200 g of SiO₂-coated gold bronze pigment (Visionaire Bright Natural Gold from ECKART GmbH & Co. KG) are dispersed with stirring in 1000 g of fully demineralized water and the dispersion is heated to 75° C. and adjusted to a pH of 6 using 10% strength hydrochloric acid. Then a solution of 9.32 g of aluminum(III) chloride in 120 g of water is added. The metering rate is 8 ml/min. The pH is kept constant by addition of 5% strength aqueous sodium hydroxide solution. Subsequently 13.6 g of carmine lake in 800 ml of fully demineralized water are introduced. The metering rate is 8 ml/min. Addition of the two solutions is followed by stirring for 30 min. The solid is separated off on a suction filter, washed free of salt with fully demineralized water, and dried in vacuo at 80° C.

This gives a red, lustrous powder.

### Example 5

### Coating of Aluminum Pigments With FeOOH

200 g of SiO₂-coated aluminum pigment (Visionaire Bright Silver Sea from ECKART GmbH & Co. KG) are dispersed with stirring in 1000 g of fully demineralized water and the dispersion is heated to 75° C. and adjusted to a pH of 4.1 using 2.5% strength hydrochloric acid. Then 101.6 g of an aqueous 40% strength iron(III) chloride solution are added. The metering rate is 2 ml/min. The pH is kept constant by addition of 5% strength aqueous sodium hydroxide solution. Addition of the two solutions is followed by stirring for 30 min. The solid is separated off on a suction filter, washed free of salt with fully demineralized water, and dried in vacuo at 110° C.

This gives a golden yellow, lustrous powder.

### Example 6

### Coating of Aluminum Pigments With FeOOH and Prussian Blue

200 g of the pigment from example 5 are dispersed with stirring in 1000 g of fully demineralized water and the dispersion is heated to 75° C. and adjusted to a pH of 4.1 using 2.5% strength hydrochloric acid. Then a solution of 28.5 g of iron(III) chloride in 160 g of water and a solution of 33.6 g of potassium hexacyanoferrate(II) in 160 g of water are added, simultaneously but separately from one another, and the pH is kept constant by simultaneous addition of 5% strength hydrochloric acid or NaOH. The metering rates are 2 ml/min. Addition of the two solutions is followed by stirring for 30 min. The solid is separated off on a suction filter, washed free of salt with fully demineralized water, and dried in vacuo at 60° C.

This gives a green, lustrous powder.

### Example 7

### Coating of Aluminum Pigments With FeOOH and Prussian Blue

200 g of SiO₂-coated aluminum pigment are dispersed with stirring in 1000 g of fully demineralized water and the dispersion is heated to 75° C. and adjusted to a pH of 4.1 using 2.5% strength hydrochloric acid. Then a solution of 101.6 g of an aqueous 40% strength iron(III) chloride solution is added. The metering rate is 2 ml/min. The pH is kept constant by addition of 5% strength aqueous sodium hydroxide solution. Following the addition of the solution the mixture is stirred for 30 min. Then a solution of 28.5 g of iron(III) chloride in 160 g of water and a solution of 33.6 g of potassium hexacyanoferrate(II) in 160 g of water are added, simultaneously but separately, and the pH is kept constant by simultaneous addition of 5% strength aqueous sodium hydroxide solution. The metering rates are 2 ml/min. Addition of the two solutions is followed by stirring for 30 min. The solid is separated off on a suction filter, washed free of salt with fully demineralized water, and dried in vacuo at 60° C.

This gives a turquoise-green, lustrous powder.

### Example 8

### Coating of Aluminum Pigments With Carmine and Prussian Blue

50 g of the pigment from example 3 are dispersed with stirring in 617 g of fully demineralized water and the dispersion is heated to 75° C. and adjusted to a pH of 4 using 2.5% strength hydrochloric acid. Then a solution of 3.56 g of iron(III) chloride in 20 g of water and a solution of 4.2 g of potassium hexacyanoferrate(II) in 20 g of water are added, simultaneously but separately from one another, and the pH is kept constant by simultaneous addition of 5% strength aqueous sodium hydroxide solution. The metering rates are 0.5 ml/min. Addition of the two solutions is followed by stirring for 30 min. The solid is separated off on a suction filter, washed free of salt with fully demineralized water, and dried in vacuo at 60° C. This gives a violet, lustrous powder.

The colored metal pigments produced in the examples above can then be incorporated into any desired cosmetics, to give cosmetics with corresponding coloration or shading.

1. A colored, platelet-shaped metal pigment comprising a base pigment comprising metal, a layer of silicon dioxide, and, subsequently, at least one additional layer comprising one or more coloring materials, wherein the coloring material in at least one said additional layer is Prussian Blue or carmine red.

2. The colored metal pigment of claim 1, wherein the base pigment is comprised of a material selected from the group consisting of aluminum, copper, zinc, iron, gold, bronze, silver and alloys thereof.

3. The colored metal pigment of claim 1, wherein the base pigment is precoated.

4. The colored metal pigment of claim 1, wherein the base pigment is precoated with a layer of aluminum oxide.

5. The colored metal pigment of claim 1, wherein the coloring material comprises a metal oxide.

6. The colored metal pigment of claim 1, wherein the one or more coloring materials comprise mixtures or successive layers of at least one of FeOOH, Fe2O3, Prussian Blue and carmine red.

7. The colored metal pigment of claim 6, wherein the successive layers are FeOOH and Prussian Blue.

8. The colored metal pigment of claim 6, wherein the successive layers are carmine red and Prussian Blue.

9. A method of producing a colored metal pigment according to claim 1, comprising the following steps:
a. coating a base metal pigment with SiO₂,
b. dispersing the metal pigment coated in step (a) in a liquid phase,
c. adding at least one solution of at least one metal salt to generate coloring materials,
d. separating off the colored metal pigment obtained in step (c), and
e. optionally drying the colored metal pigment obtained in step (d).

10. The method of claim 9, wherein in step (c) the pH is set in the range from 1.7 to 9.5.

11. The method of claim 9, wherein the pH is kept constant by metered addition of a suitable acid or base.

12. The method of claim 9, wherein in step (a) said coating takes place using a sol-gel process.

13. The method of claim 9, wherein in step (a) waterglass is used.

14. A method of producing a cosmetic, selected from the group consisting of eyeshadows, mascara, lipsticks, makeup formulations, creams, powders, eyeliners, nail varnish, haircare compositions, soap, sun protection compositions and perfume, which comprises formulating said cosmetics with a colored metal pigment according to claim 1.

15. A cosmetic comprising colored metal pigment according to claim 1.

16. The colored metal pigment of claim 5, wherein the metal oxide is selected from the group consisting of FeOOH, Fe₂O₃, Cr₂O₃ and mixtures thereof.

17. The method of claim 9, wherein water is selected as the liquid phase.
